(19) United States
(12) Patent Application Publication
(10) Pub. No.: US 2026/0137747 A1
(43) Pub. Date: May 21, 2026

(54) CHINESE MEDICINE COMPOSITION FOR TREATING BREAST CANCER AND METHOD FOR TREATING BREAST CANCER USING THE SAME

(71) Applicant: YU-TE LIU, Taichung City (TW)

(72) Inventors: YU-TE LIU, Taichung City (TW); TSAI-CHING HSU, Taichung City (TW); BOR-SHOW TZANG, Taichung City (TW)

(73) Assignee: YU-TE LIU, Taichung City (TW)

(21) Appl. No.: 19/049,857

(22) Filed: Feb. 10, 2025

(30) Foreign Application Priority Data
Nov. 15, 2024 (TW) 113144134

(51) Int. Cl. A61K 36/65; A61K 36/288; A61K 36/47; A61K 36/484; A61K 36/744; A61K 36/8998; A61P 35/00

(52) U.S. Cl. CPC A61K 36/65; A61K 36/288; A61K 36/47; A61K 36/484; A61K 36/744; A61K 36/8998; A61P 35/00

(57) ABSTRACT

A Chinese medicine composition for treating breast cancer includes Euphorbia kansui, Paeonia lactiflora, Glycyrrhiza uralensis, Taraxacum, Gardeniae fructus, and Hordeum vulgare. A weight ratio of Euphorbia kansui, Paeonia lactiflora, Glycyrrhiza uralensis, Taraxacum, Gardeniae fructus, and Hordeum vulgare in the Chinese medicine composition is 1-2:4-6:2-3:3-5:2-4:2-3. Moreover, a method for treating breast cancer using the Chinese medicine composition is provided, including a step of administering the Chinese medicine composition at an effective dose ranging from 0.1 mg/ml to 10.0 mg/ml to a plurality of breast cancer cells for cultivation. Cell tests have confirmed that the Chinese medicine composition inhibits the activity of the breast cancer cells, achieving the purpose of treating breast cancer symptoms or inhibiting the activity of the breast cancer cells.

### BACKGROUND OF THE INVENTION

### Technical Field

The present invention relates generally to a traditional Chinese medicine, and more particularly to a Chinese medicine composition for treating breast cancer and a method for treating breast cancer using the Chinese medicine composition.

### Description of Related Art

Breast cancer is one of the most common malignant tumors in women. The main treatments for breast cancer currently include breast surgery, radiation therapy, and targeted drug therapy. Taking targeted drug therapy as an example, the targeted drug therapy involves administering targeted drugs to the patient to inhibit the growth of breast cancer cells, slow down the spread of the breast cancer cells, and prevent cancer recurrence.

However, the targeted drugs currently used in targeted drug therapy for cancer could have significant side effects on the human body. The existing targeted drug for breast cancer is tamoxifen. Tamoxifen is a hormone inhibitor that works by binding to estrogen receptors, preventing the transmission of cellular signals and achieving tumor treatment effects. However, long-term use of tamoxifen could increase the risk of endometrial cancer and venous thrombosis in a patient, and might even lead to the development of drug resistance in breast cancer cells, ultimately resulting in treatment failure.

In order to improve the side effects of breast cancer treatments, some related companies have developed natural Chinese herbal medicines as adjunctive treatments. However, current research has not yet successfully developed a Chinese medicine composition formula that is effective in inhibiting drug-resistant breast cancer cells. Therefore, the technical challenge of selecting an appropriate Chinese medicine composition formula to effectively treat or inhibit breast cancer symptoms is the objective that this invention aims to address.

### BRIEF SUMMARY OF THE INVENTION

In view of the above, the primary objective of the present invention is to provide a Chinese medicine composition for treating breast cancer and a method for treating breast cancer using the Chinese medicine composition. The Chinese medicine composition includes Euphorbia kansui, Paeonia lactiflora, Glycyrrhiza uralensis, Taraxacum, Gardeniae fructus, and Hordeum vulgare. Cell tests have confirmed that the Chinese medicine composition inhibits the activity of conventional breast cancer cells and drug-resistant breast cancer cells.

The present invention provides a Chinese medicine composition for treating breast cancer, including Euphorbia kansui, Paeonia lactiflora, Glycyrrhiza uralensis, Taraxacum, Gardeniae fructus, and Hordeum vulgare. A weight ratio of Euphorbia kansui, Paeonia lactiflora, Glycyrrhiza uralensis, Taraxacum, Gardeniae fructus, and Hordeum vulgare in the Chinese medicine composition is 1-2:4-6:2-3:3-5:2-4:2-3.

The present invention also provides a method for treating breast cancer using a Chinese medicine composition, including a step of administering the Chinese medicine composition at an effective dose ranging from 0.1 mg/ml to 10.0 mg/ml to a plurality of breast cancer cells for cultivation. The Chinese medicine composition includes Euphorbia kansui, Paeonia lactiflora, Glycyrrhiza uralensis, Taraxacum, Gardeniae fructus, and Hordeum vulgare. The Chinese medicine composition is for inhibiting an activity of the breast cancer cells. The breast cancer cells are selected from a group consisting of conventional breast cancer cells (MCF-7), tamoxifen-resistant breast cancer cells (MCF-7/TAMR1), and tamoxifen-sensitive breast cancer cells (MCF-7/S0.5).

In summary, the weight ratios of Euphorbia kansui, Paeonia lactiflora, Glycyrrhiza uralensis, Taraxacum, Gardeniae fructus, and Hordeum vulgare in the Chinese medicine composition have been validated through the cell tests to effectively inhibit or reduce the activity of the breast cancer cells. The effective dose of the Chinese medicine composition, ranging from 0.1 to 10.0 mg/ml, could effectively inhibit the activity of the conventional breast cancer cells (MCF-7), the tamoxifen-resistant breast cancer cells (MCF-7/TAMR1), and the tamoxifen-sensitive breast cancer cells (MCF-7/S 0.5) under at least 24 hours of cultivation time. Correspondingly, the effective dose of the Chinese medicine composition, ranging from 0.1 to 10.0 mg/ml, reduces the number of the conventional breast cancer cells (MCF-7), the tamoxifen-resistant breast cancer cells (MCF-7/TAMR1), and the tamoxifen-sensitive breast cancer cells (MCF-7/S0.5). When the breast cancer cells are the conventional breast cancer cells (MCF-7), the effective dose of the Chinese medicine composition, ranging from 5.0 to 10.0 mg/ml, effectively inhibits the activity of the conventional breast cancer cells (MCF-7). When the breast cancer cells are the tamoxifen-resistant breast cancer cells (MCF-7/TAMR1), the effective dose of the Chinese medicine composition, ranging from 1.25 to 10.0 mg/ml, effectively inhibits the activity of the tamoxifen-resistant breast cancer cells (MCF-7/TAMR1). When the breast cancer cells are the tamoxifen-sensitive breast cancer cells (MCF-7/S0.5), the effective dose of the Chinese medicine composition, ranging from 2.5 to 10.0 mg/ml, effectively inhibits the activity of the tamoxifen-sensitive breast cancer cells (MCF-7/S0.5). Therefore, the composition ratio of the Chinese medicine composition, when administered at the specific effective dose, achieves the goal of treating breast cancer symptoms or inhibiting the activity of breast cancer cells.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1A is a chart having cell images, showing the conventional breast cancer cells (MCF-7) that are respectively administered with the Chinese medicine composition in the experimental groups 1-6 at effective doses of 0.1 mg/ml, 0.5 mg/ml, 1.25 mg/ml, 2.5 mg/ml, 5 mg/ml, and 10 mg/ml, and cultured for 24, 48, and 72 hours;

FIG. 1B is a bar chart, showing the cell viability data of the conventional breast cancer cells (MCF-7) that are respectively administered with the Chinese medicine composition in the experimental groups 1-6 at effective doses of 0.1 mg/ml, 0.5 mg/ml, 1.25 mg/ml, 2.5 mg/ml, 5 mg/ml, and 10 mg/ml, and cultured for 24 hours;

FIG. 1C is a bar chart, showing the cell viability data of the conventional breast cancer cells (MCF-7) that are respectively administered with the Chinese medicine composition in the experimental groups 1-6 at effective doses of 0.1 mg/ml, 0.5 mg/ml, 1.25 mg/ml, 2.5 mg/ml, 5 mg/ml, and 10 mg/ml, and cultured for 48 hours;

FIG. 1D is a bar chart, showing the cell viability data of the conventional breast cancer cells (MCF-7) that are respectively administered with the Chinese medicine composition in the experimental groups 1-6 at effective doses of 0.1 mg/ml, 0.5 mg/ml, 1.25 mg/ml, 2.5 mg/ml, 5 mg/ml, and 10 mg/ml, and cultured for 72 hours;

FIG. 2A is a chart having cell images, showing the tamoxifen-resistant breast cancer cells (MCF-7/TAMR1) that are respectively administered with the Chinese medicine composition in the experimental groups 7-12 at effective doses of 0.1 mg/ml, 0.5 mg/ml, 1.25 mg/ml, 2.5 mg/ml, 5 mg/ml, and 10 mg/ml, and cultured for 24, 48, and 72 hours;

FIG. 2B is a bar chart, showing the cell viability data of the tamoxifen-resistant breast cancer cells (MCF-7/TAMR1) that are respectively administered with the Chinese medicine composition in the experimental groups 7-12 at effective doses of 0.1 mg/ml, 0.5 mg/ml, 1.25 mg/ml, 2.5 mg/ml, 5 mg/ml, and 10 mg/ml, and cultured for 24 hours;

FIG. 2C is a bar chart, showing the cell viability data of the tamoxifen-resistant breast cancer cells (MCF-7/TAMR1) that are respectively administered with the Chinese medicine composition in the experimental groups 7-12 at effective doses of 0.1 mg/ml, 0.5 mg/ml, 1.25 mg/ml, 2.5 mg/ml, 5 mg/ml, and 10 mg/ml, and cultured for 48 hours;

FIG. 2D is a bar chart, showing the cell viability data of the tamoxifen-resistant breast cancer cells (MCF-7/TAMR1) that are respectively administered with the Chinese medicine composition in the experimental groups 7-12 at effective doses of 0.1 mg/ml, 0.5 mg/ml, 1.25 mg/ml, 2.5 mg/ml, 5 mg/ml, and 10 mg/ml, and cultured for 72 hours;

FIG. 3A is a chart having cell images, showing the migrated tamoxifen-resistant breast cancer cells (MCF-7/TAMR1) that are respectively administered with the Chinese medicine composition in the experimental groups 7-12 at effective doses of 0.1 mg/ml, 0.5 mg/ml, 1.25 mg/ml, 2.5 mg/ml, 5 mg/ml, and 10 mg/ml;

FIG. 3B is a bar chart, showing the cell migration data of the tamoxifen-resistant breast cancer cells (MCF-7/TAMR1) that are respectively administered with the Chinese medicine composition in the experimental groups 7-12 at effective doses of 0.1 mg/ml, 0.5 mg/ml, 1.25 mg/ml, 2.5 mg/ml, 5 mg/ml, and 10 mg/ml, and cultured for 24 hours;

FIG. 4A is a chart having cell images, showing the tamoxifen-resistant breast cancer cells (MCF-7/TAMR1) that are respectively administered with the Chinese medicine composition in the experimental groups 7-12 at effective doses of 0.1 mg/ml, 0.5 mg/ml, 1.25 mg/ml, 2.5 mg/ml, 5 mg/ml, and 10 mg/ml, wherein the cell images are used to observe the effects on cell invasion;

FIG. 4B is a bar chart, showing the number of the tamoxifen-resistant breast cancer cells (MCF-7/TAMR1) that are respectively administered with the Chinese medicine composition in the experimental groups 7-12 at effective doses of 0.1 mg/ml, 0.5 mg/ml, 1.25 mg/ml, 2.5 mg/ml, 5 mg/ml, and 10 mg/ml, which underwent cell invasion and subsequent migration;

FIG. 5A is a protein electrophoresis gel image, showing the expression of ATG5, ATG7, and LC3-II proteins of the tamoxifen-resistant breast cancer cells (MCF-7/TAMR1) that are respectively administered with the Chinese medicine composition in the experimental groups 7-12 at effective doses of 0.1 mg/ml, 0.5 mg/ml, 1.25 mg/ml, 2.5 mg/ml, 5 mg/ml, and 10 mg/ml;

FIG. 5B is a bar chart, showing the expression of ATG5, ATG7, and LC3-II proteins of the tamoxifen-resistant breast cancer cells (MCF-7/TAMR1) that are respectively administered with the Chinese medicine composition in the experimental groups 7-12 at effective doses of 0.1 mg/ml, 0.5 mg/ml, 1.25 mg/ml, 2.5 mg/ml, 5 mg/ml, and 10 mg/ml;

FIG. 6A is a chart having cell images, showing the tamoxifen-sensitive breast cancer cells (MCF-7/S0.5) that are respectively administered with the Chinese medicine composition in the experimental groups 13-18 at effective doses of 0.1 mg/ml, 0.5 mg/ml, 1.25 mg/ml, 2.5 mg/ml, 5 mg/ml, and 10 mg/ml, and cultured for 24, 48, and 72 hours;

FIG. 6B is a bar chart, showing the cell viability data of the tamoxifen-sensitive breast cancer cells (MCF-7/S0.5) that are respectively administered with the Chinese medicine composition in the experimental groups 13-18 at effective doses of 0.1 mg/ml, 0.5 mg/ml, 1.25 mg/ml, 2.5 mg/ml, 5 mg/ml, and 10 mg/ml, and cultured for 24 hours;

FIG. 6C is a bar chart, showing the cell viability data of the tamoxifen-sensitive breast cancer cells (MCF-7/S0.5) that are respectively administered with the Chinese medicine composition in the experimental groups 13-18 at effective doses of 0.1 mg/ml, 0.5 mg/ml, 1.25 mg/ml, 2.5 mg/ml, 5 mg/ml, and 10 mg/ml, and cultured for 48 hours;

FIG. 6D is a bar chart, showing the cell viability data of the tamoxifen-sensitive breast cancer cells (MCF-7/S0.5) that are respectively administered with the Chinese medicine composition in the experimental groups 13-18 at effective doses of 0.1 mg/ml, 0.5 mg/ml, 1.25 mg/ml, 2.5 mg/ml, 5 mg/ml, and 10 mg/ml, and cultured for 72 hours;

FIG. 7A is a chart having cell images, showing the migrated tamoxifen-sensitive breast cancer cells (MCF-7/S0.5) that are respectively administered with the Chinese medicine composition in the experimental groups 13-18 at effective doses of 0.1 mg/ml, 0.5 mg/ml, 1.25 mg/ml, 2.5 mg/ml, 5 mg/ml, and 10 mg/ml;

FIG. 7B is a bar chart, showing the cell migration data of the tamoxifen-sensitive breast cancer cells (MCF-7/S0.5) that are respectively administered with the Chinese medicine composition in the experimental groups 13-18 at effective doses of 0.1 mg/ml, 0.5 mg/ml, 1.25 mg/ml, 2.5 mg/ml, 5 mg/ml, and 10 mg/ml, and cultured for 24 hours;

FIG. 8A is a chart having cell images, showing the tamoxifen-sensitive breast cancer cells (MCF-7/S0.5) that are respectively administered with the Chinese medicine composition in the experimental groups 13-18 at effective doses of 0.1 mg/ml, 0.5 mg/ml, 1.25 mg/ml, 2.5 mg/ml, 5 mg/ml, and 10 mg/ml, wherein the cell images are used to observe the effects on cell invasion;

FIG. 8B is a bar chart, showing the number of the tamoxifen-sensitive breast cancer cells (MCF-7/S0.5) that are respectively administered with the Chinese medicine composition in the experimental groups 13-18 at effective doses of 0.1 mg/ml, 0.5 mg/ml, 1.25 mg/ml, 2.5 mg/ml, 5 mg/ml, and 10 mg/ml, which underwent cell invasion and subsequent migration;

FIG. 9A is a protein electrophoresis gel image, showing the expression of ATG5, ATG7, and LC3-II proteins of the tamoxifen-sensitive breast cancer cells (MCF-7/S0.5) that are respectively administered with the Chinese medicine composition in the experimental groups 13-18 at effective doses of 0.1 mg/ml, 0.5 mg/ml, 1.25 mg/ml, 2.5 mg/ml, 5 mg/ml, and 10 mg/ml;

FIG. 9B is a bar chart, showing the expression of ATG5, ATG7, and LC3-II proteins of the tamoxifen-sensitive breast cancer cells (MCF-7/S0.5) that are respectively administered with the Chinese medicine composition in the experimental groups 13-18 at effective doses of 0.1 mg/ml, 0.5 mg/ml, 1.25 mg/ml, 2.5 mg/ml, 5 mg/ml, and 10 mg/ml.

### DETAILED DESCRIPTION OF THE INVENTION

A Chinese medicine composition for treating breast cancer, according to an embodiment of the present invention, is adapted to reduce or inhibit an activity of a plurality of breast cancer cells and includes Euphorbia kansui, Paeonia lactiflora, Glycyrrhiza uralensis, Taraxacum, Gardeniae Fructus, and Hordeum vulgare. The Euphorbia kansui, Paeonia lactiflora, Glycyrrhiza uralensis, Taraxacum, Gardeniae Fructus, and Hordeum vulgare are obtained through extraction of corresponding natural plants.

The pharmacological characteristics of each of the ingredients in the Chinese medicine composition are described in detail below:
Euphorbia kansui (Gansui): The active components of Euphorbia kansui include diterpenoids and triterpenoids. Diterpenoids include ingenol derivatives and jatrophanes. Ingenol derivatives exhibit significant anticancer and antiviral activities. Triterpenoids include euphol and tirucallol. Triterpenoids also have anticancer properties.

Paeonia lactiflora (Baishao): The active components of Paeonia lactiflora include paeoniflorins and their derivatives, flavonoids, organic acids, and so on. Paeoniflorins are the main active component of Paeonia lactiflora and have various pharmacological effects, including anti-inflammatory, analgesic, antioxidant, and immune-regulatory functions.

Glycyrrhiza uralensis (Gancao): The active components of Glycyrrhiza uralensis include flavonoids, triterpenoid saponins, and glycyrrhiza polysaccharides (GPS). The active components of Glycyrrhiza uralensis have shown good effects in clinical trials, including anti-inflammatory, antitumor, liver protection, and immune-enhancing properties.

Taraxacum (Pugongying): The active components of Taraxacum include flavonoids, sterols, phenols, choline, and organic acids. The active components of Taraxacum have demonstrated various effects in clinical trials, including liver protection, antithrombotic activity, blood sugar reduction, anti-inflammatory, antioxidant, antibacterial, and antitumor properties.

Gardeniae fructus (Zhizi): The active components of Gardeniae fructus include iridoids, monoterpene glucosides, diterpenoids, triterpenoids, volatile oils, organic acid esters, flavonoids, polysaccharides, and trace elements. The active components of Gardeniae fructus have demonstrated anti-inflammatory, antithrombotic, antiviral, and antitumor effects in clinical studies.

Hordeum vulgare (Maiya): The active components of Hordeum vulgare include α-amylase, β-amylase, catalytic enzymes, maltose, hordenine, adenine, choline, proteins, amino acids, vitamins B, D, E, cytochrome C, and so on. The active components of Hordeum vulgare have digestive aid, blood sugar reduction, and antimicrobial effects.

A weight ratio of Euphorbia kansui, Paeonia lactiflora, Glycyrrhiza uralensis, Taraxacum, Gardeniae fructus, and Hordeum vulgare in the Chinese medicine composition is 1-2□4-6□2-3□3-5□2-4□2-3. The weight ratio of Euphorbia kansui, Paeonia lactiflora, Glycyrrhiza uralensis, Taraxacum, Gardeniae fructus, and Hordeum vulgare in the Chinese medicine composition could be adjusted according to the spread of the breast cancer cells.

In one embodiment, during actual clinical research on the Chinese medicine composition, if a patient experiences severe tumor pain due to the breast cancer cells significantly compressing other organs, adjusting the weight ratio of Euphorbia kansui, Paeonia lactiflora, Glycyrrhiza uralensis, Taraxacum, Gardeniae Fructus, and Hordeum vulgare in the Chinese medicine composition to 1:6:3:3:2:2 and regularly administering the Chinese medicine composition to the patient could effectively alleviate the symptoms of breast cancer in the patient.

In another embodiment, during actual clinical research on the Chinese medicine composition, if the patient develops severe lymphedema after surgery, adjusting the weight ratio of Euphorbia kansui, Paeonia lactiflora, Glycyrrhiza uralensis, Taraxacum, Gardeniae Fructus, and Hordeum vulgare in the Chinese medicine composition to 2:4:2:3:2:3 and regularly administering the Chinese medicine composition to the postoperative patient could not only inhibit and reduce the activity of breast cancer cells but also improve the symptoms of lymphedema.

In yet another embodiment, during actual clinical research on the Chinese medicine composition, if the patient experiences severe skin toxicity during chemotherapy, adjusting the weight ratio of Euphorbia kansui, Paeonia lactiflora, Glycyrrhiza uralensis, Taraxacum, Gardeniae Fructus, and Hordeum vulgare in the Chinese medicine composition to 1:4:2:5:4:2 and regularly administering the Chinese medicine composition to the patient undergoing chemotherapy could not only inhibit and reduce the activity of breast cancer cells but also effectively alleviate chemotherapy-related skin toxicity reactions.

Moreover, a method for treating breast cancer using the Chinese medicine composition according to the present invention is provided. The method for treating breast cancer using the Chinese medicine composition includes a step of:
Administering the Chinese medicine composition at an effective dose ranging from 0.1 mg/ml to 10.0 mg/ml to the breast cancer cells for cultivation.

In the current embodiment, the Chinese medicine composition is administered to the breast cancer cells for a cultivation time ranging from 24 to 72 hours. The breast cancer cells are selected from a group consisting of conventional breast cancer cells (MCF-7), tamoxifen-resistant breast cancer cells (MCF-7/TAMR1), and tamoxifen-sensitive breast cancer cells (MCF-7/S0.5). The Chinese medicine composition inhibits the activity of the breast cancer cells, thereby achieving the purpose of treating breast cancer symptoms or reducing breast cancer cell activity.

In order to demonstrate the purpose, the features, and the effects of the present invention, a plurality of cell tests were conducted by respectively administering the Chinese medicine composition of the embodiment at various effective doses to the conventional breast cancer cells (MCF-7), the tamoxifen-resistant breast cancer cells (MCF-7/TAMR1), and the tamoxifen-sensitive breast cancer cells (MCF-7/S0.5). The cell viability of the conventional breast cancer cells, the tamoxifen-resistant breast cancer cells, and the tamoxifen-sensitive breast cancer cells was analyzed at different cultivation times post-administration. The conventional breast cancer cells (MCF-7), the tamoxifen-resistant breast cancer cells (MCF-7/TAMR1), and the tamoxifen-sensitive breast cancer cells (MCF-7/SO.5) were purchased from Merck Millipore Corporation. In the cell tests, the weight ratio of Euphorbia kansui, Paeonia lactiflora, Glycyrrhiza uralensis, Taraxacum, Gardeniae Fructus, and Hordeum vulgare in the Chinese medicine composition is 1:6:3:3:2:2. This demonstrates that the Chinese medicine composition provides an effective inhibitory or reducing effect on breast cancer cell activity.

In the following embodiments, an applicable range of the present invention could be proved, and the skilled person in the art could properly adjusted the parameters by referring to the following embodiments, wherein the applicable range which is properly adjusted by the skilled person in the art is still in the scope of the present invention.

The following investigates the cell viability of the conventional breast cancer cells (MCF-7) after administering the Chinese medicine composition:

1. The groups in the current test include control group 1 and experimental groups 1 to 6.

Control group 1: The control group 1 includes the conventional breast cancer cells (MCF-7) that were not administered the Chinese medicine composition. An effective dose of the Chinese medicine composition is 0 mg/ml. The conventional breast cancer cells (MCF-7) were cultured for 24, 48, and 72 hours, respectively.

Experimental group 1: The experimental group 1 includes the conventional breast cancer cells (MCF-7) that were administered the Chinese medicine composition at an effective dose of 0.1 mg/ml. The conventional breast cancer cells (MCF-7) were cultured for 24, 48, and 72 hours, respectively, following the administration.

Experimental group 2: The experimental group 2 includes the conventional breast cancer cells (MCF-7) that were administered the Chinese medicine composition at an effective dose of 0.5 mg/ml. The conventional breast cancer cells (MCF-7) were cultured for 24, 48, and 72 hours, respectively, following the administration.

Experimental group 3: The experimental group 3 includes the conventional breast cancer cells (MCF-7) that were administered the Chinese medicine composition at an effective dose of 1.25 mg/ml. The conventional breast cancer cells (MCF-7) were cultured for 24, 48, and 72 hours, respectively, following the administration.

Experimental group 4: The experimental group 4 includes the conventional breast cancer cells (MCF-7) that were administered the Chinese medicine composition at an effective dose of 2.5 mg/ml. The conventional breast cancer cells (MCF-7) were cultured for 24, 48, and 72 hours, respectively, following the administration.

Experimental group 5: The experimental group 5 includes the conventional breast cancer cells (MCF-7) that were administered the Chinese medicine composition at an effective dose of 5.0 mg/ml. The conventional breast cancer cells (MCF-7) were cultured for 24, 48, and 72 hours, respectively, following the administration.

Experimental group 6: The experimental group 6 includes the conventional breast cancer cells (MCF-7) that were administered the Chinese medicine composition at an effective dose of 10.0 mg/ml. The conventional breast cancer cells (MCF-7) were cultured for 24, 48, and 72 hours, respectively, following the administration.

2. Cell viability analysis of the conventional breast cancer cells (MCF-7) in each of the experimental groups 1-6:
The test includes the following steps: culturing the conventional breast cancer cells (MCF-7) in three microplates, wherein each microplate has 96 wells; plating a total of 5×10³ conventional breast cancer cells (MCF-7) into each well and culturing with cell culture medium for 24 hours; removing the cell culture medium after the conventional breast cancer cells (MCF-7) have adhered to the corresponding wells; injecting the effective doses of the Chinese medicine composition prepared for the control group 1 and experimental groups 1-6 into the wells of each microplate that has the conventional breast cancer cells (MCF-7), respectively; placing the three microplates into cell culture with cell culture medium for 24 hours, 48 hours, and 72 hours, respectively; observing and recording the cell count using an electron microscope during the 24-hour, 48-hour, and 72-hour cultivation times for the control group 1 and the experimental groups 1-6; removing the cell culture medium; washing the wells with sterilized phosphate-buffered saline (PBS) for two times; adding 0.2 ml of cell culture medium to the wells; placing the microplates into an incubator for 2 hours; removing the cell culture medium; washing the wells with unsterilized phosphate-buffered saline (PBS) for one time; adding 0.2 mL DMSO into each well to dissolve crystals; and using an enzyme-linked immuno-sorbent assay (ELISA) reader to measure the optical density (OD) value at a wavelength of 570 nm, thereby calculating the relative cell viability.

The cell viability data measured for the experimental groups 1-6 are calculated by setting the absorbance of the control group 1 as 100%. The absorbance of each of the experimental groups 1-6 is then divided by the absorbance of the control group 1 to determine the relative cell viability of each experimental group compared to the control group 1.

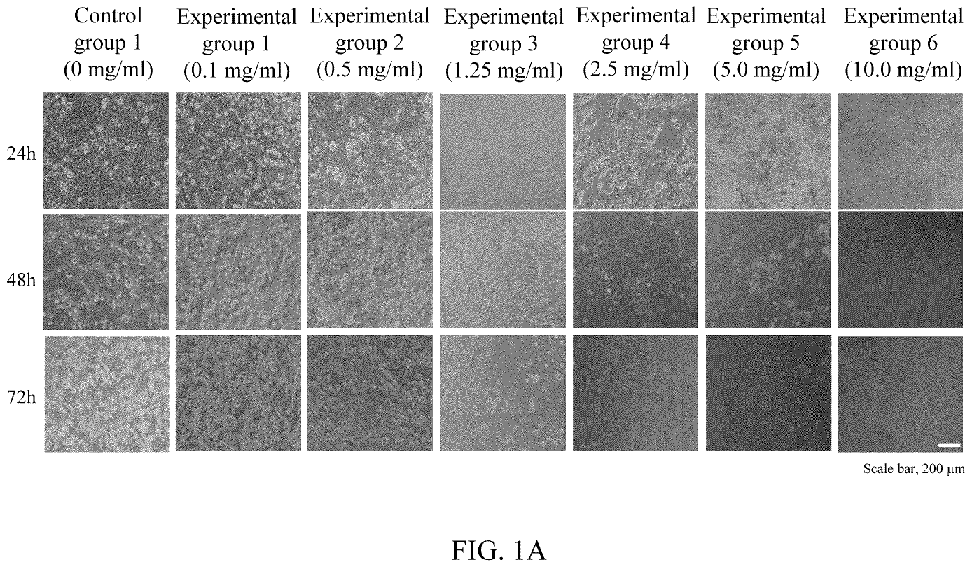

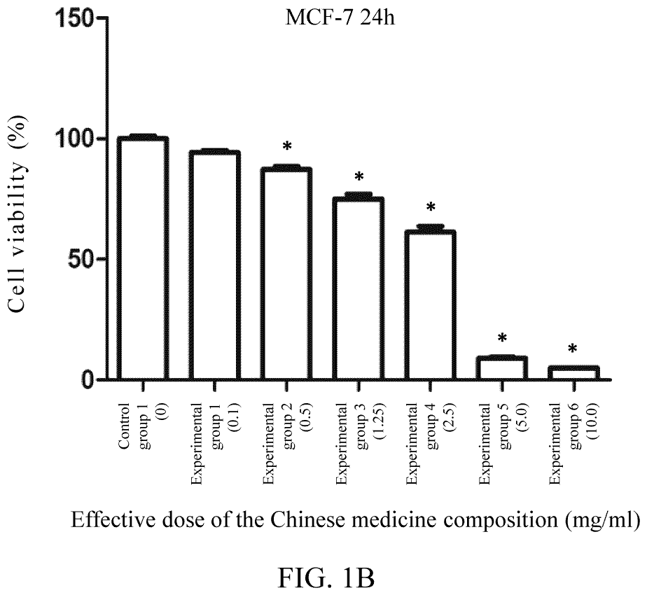

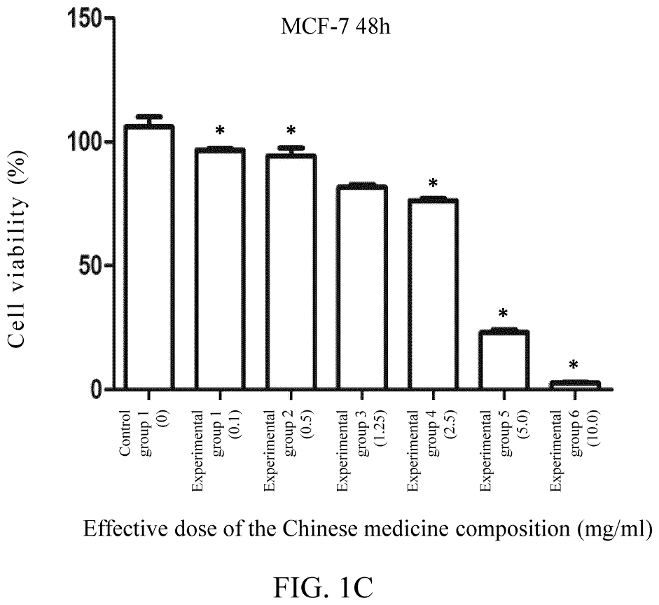

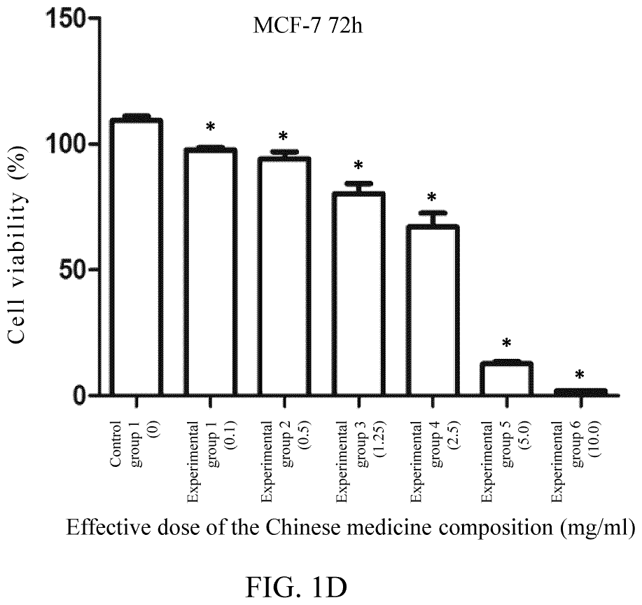

The observation results are shown in FIG. 1A, FIG. 1B, FIG. 1C, and FIG. 1D. FIG. 1A is a chart displaying a plurality of cell images, showing the conventional breast cancer cells (MCF-7) in the experimental groups 1-6, corresponding to 24-hour, 48-hour, and 72-hour cultivation times. FIG. 1B, FIG. 1C, and FIG. 1D respectively present bar charts, showing cell viability data of the conventional breast cancer cells (MCF-7) in the experimental groups 1-6, corresponding to 24-hour, 48-hour, and 72-hour cultivation times. In FIG. 1B to FIG. 1D, the Standard Error (SE) for the experimental examples 1-6 is shown, which were obtained from three independent tests (n=3) conducted for each of the experimental groups 1-6. The symbol “*” indicates a statistically significant comparison between the experimental groups 1-6 and the control group 1, with p<0.05.

As shown in FIG. 1A and FIG. 1B, based on the results from the experimental groups 1-6 under 24-hour cultivation time, the cell viability of the conventional breast cancer cells (MCF-7) in the experimental groups 1 and 2 did not show a significant decrease compared to the control group 1. The cell viability of the conventional breast cancer cells (MCF-7) in the experimental groups 3 and 4 showed a gradual decrease compared to the control group 1. This indicates that administering the effective doses of 1.25 mg/ml and 2.5 mg/ml of the Chinese medicine composition in the experimental groups 5 and 6, respectively, could appropriately inhibit or reduce the growth of the conventional breast cancer cells (MCF-7). The cell viability of the conventional breast cancer cells (MCF-7) in the experimental groups 5 and 6 was significantly reduced to below 10% compared to the control group 1, wherein the cell viability of the conventional breast cancer cells (MCF-7) in the experimental group 6 was further reduced compared to the experimental group 5. As shown in FIG. 1A, the cell images of the experimental groups 5 and 6 showed a significant reduction in cell density compared to the experimental groups 1-4. This indicates that administering the effective doses of 5.0 mg/ml and 10.0 mg/ml of the Chinese medicine composition under 24-hour cultivation time in the experimental groups 5 and 6, respectively, effectively reduced or inhibited the growth of the conventional breast cancer cells (MCF-7). Moreover, the effective dose of 10.0 mg/ml administered in the experimental group 6 exhibited a more pronounced effect in inhibiting the growth of the conventional breast cancer cells (MCF-7) compared to the experimental group 5.

As shown in FIG. 1A and FIG. 1C, based on the results from the experimental groups 1-6 under 48-hour cultivation time, the cell viability of the conventional breast cancer cells (MCF-7) in the experimental groups 1 and 2 also did not show a significant decrease compared to the control group 1. The cell viability of the conventional breast cancer cells (MCF-7) in the experimental groups 3 and 4 showed a gradual decrease compared to the control group 1. As shown in FIG. 1B and FIG. 1C, the cell viability of the conventional breast cancer cells (MCF-7) in experimental groups 3 and 4 did not significantly decrease after 48 hours of cultivation. However, the cell viability of the conventional breast cancer cells (MCF-7) in the experimental groups 5 and 6 was similarly reduced to below 10% compared to the control group 1. The cell viability of the conventional breast cancer cells (MCF-7) in the experimental group 6 was further reduced after 48-hour cultivation time compared to 24-hour cultivation time.

As shown in FIG. 1A and FIG. 1D, based on the results from the experimental groups 1-6 under 72-hour cultivation time, the cell viability of the conventional breast cancer cells (MCF-7) in the experimental groups 1-4 showed a decreasing trend compared to the control group 1. The cell viability of the conventional breast cancer cells (MCF-7) in the experimental groups 5 and 6 was similarly reduced to below 10% compared to the control group 1. As shown in FIG. 1C and FIG. 1D, the cell viability of the conventional breast cancer cells (MCF-7) in the experimental group 6 was further reduced after 72-hour cultivation time compared to 24-hour cultivation time.

Thus, the effective doses of the Chinese medicine composition ranging from 0.1 to 10.0 mg/ml could effectively inhibite or reduce the activity of the conventional breast cancer cells (MCF-7). The effective doses ranging from 1.25 to 10.0 mg/ml could significantly reduce the cell viability of the the conventional breast cancer cells. The effective doses of the Chinese medicine composition ranging from 5.0 to 10.0 mg/ml more significantly inhibit the activity of conventional breast cancer cells (MCF-7).

The following investigates the cell viability of the tamoxifen-resistant breast cancer cells (MCF-7/TAMR1) after administering the Chinese medicine composition:

1. The groups in the current test include control group 2 and experimental groups 7 to 12.

Control group 2: The control group 2 includes the tamoxifen-resistant breast cancer cells (MCF-7/TAMR1) that were not administered the Chinese medicine composition. An effective dose of the Chinese medicine composition is 0 mg/ml. The tamoxifen-resistant breast cancer cells (MCF-7/TAMR 1) were cultured for 24, 48, and 72 hours, respectively.

Experimental group 7: The experimental group 7 includes the tamoxifen-resistant breast cancer cells (MCF-7/TAMR1) that were administered the Chinese medicine composition at an effective dose of 0.1 mg/ml. The tamoxifen-resistant breast cancer cells (MCF-7/TAMR 1) were cultured for 24, 48, and 72 hours, respectively, following the administration.

Experimental group 8: The experimental group 8 includes the tamoxifen-resistant breast cancer cells (MCF-7/TAMR1) that were administered the Chinese medicine composition at an effective dose of 0.5 mg/ml. The tamoxifen-resistant breast cancer cells (MCF-7/TAMR 1) were cultured for 24, 48, and 72 hours, respectively, following the administration.

Experimental group 9: The experimental group 9 includes the tamoxifen-resistant breast cancer cells (MCF-7/TAMR1) that were administered the Chinese medicine composition at an effective dose of 1.25 mg/ml. The tamoxifen-resistant breast cancer cells (MCF-7/TAMR 1) were cultured for 24, 48, and 72 hours, respectively, following the administration.

Experimental group 10: The experimental group 10 includes the tamoxifen-resistant breast cancer cells (MCF-7/TAMR1) that were administered the Chinese medicine composition at an effective dose of 2.5 mg/ml. The tamoxifen-resistant breast cancer cells (MCF-7/TAMR 1) were cultured for 24, 48, and 72 hours, respectively, following the administration.

Experimental group 11: The experimental group 11 includes the tamoxifen-resistant breast cancer cells (MCF-7/TAMR1) that were administered the Chinese medicine composition at an effective dose of 5.0 mg/ml. The tamoxifen-resistant breast cancer cells (MCF-7/TAMR 1) were cultured for 24, 48, and 72 hours, respectively, following the administration.

Experimental group 12: The experimental group 12 includes the tamoxifen-resistant breast cancer cells (MCF-7/TAMR1) that were administered the Chinese medicine composition at an effective dose of 10.0 mg/ml. The tamoxifen-resistant breast cancer cells (MCF-7/TAMR 1) were cultured for 24, 48, and 72 hours, respectively, following the administration.

2. Cell viability analysis of the tamoxifen-resistant breast cancer cells (MCF-7/TAMR 1) in each of the experimental groups 7-12:
The test steps for cell viability of the tamoxifen-resistant breast cancer cells (MCF-7/TAMR1) in the control group 2 and the experimental groups 7-12 are the same as the test steps for the control group 1 and the experimental groups 1-6. Therefore, the test steps for cell viability of the tamoxifen-resistant breast cancer cells (MCF-7/TAMR1) are not reiterated here.

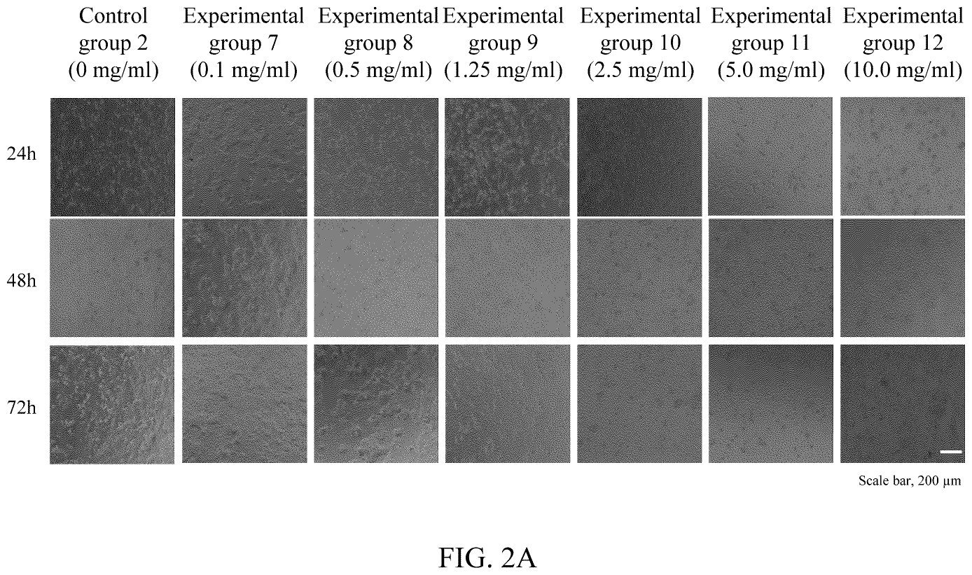

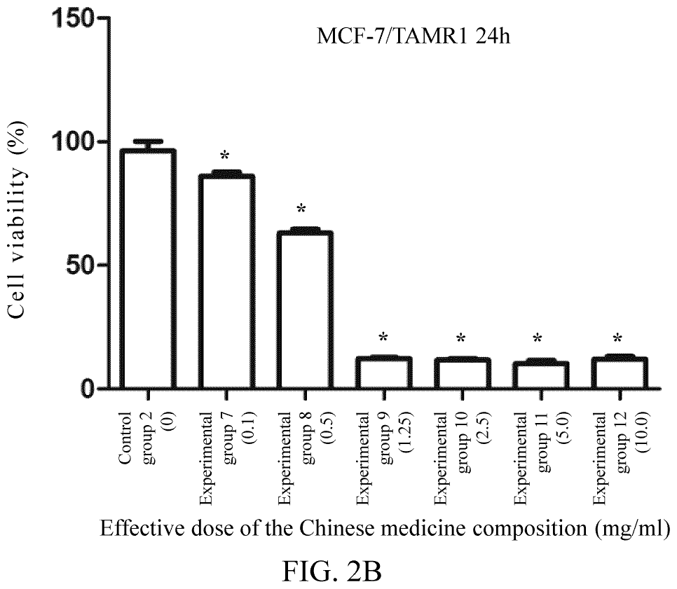

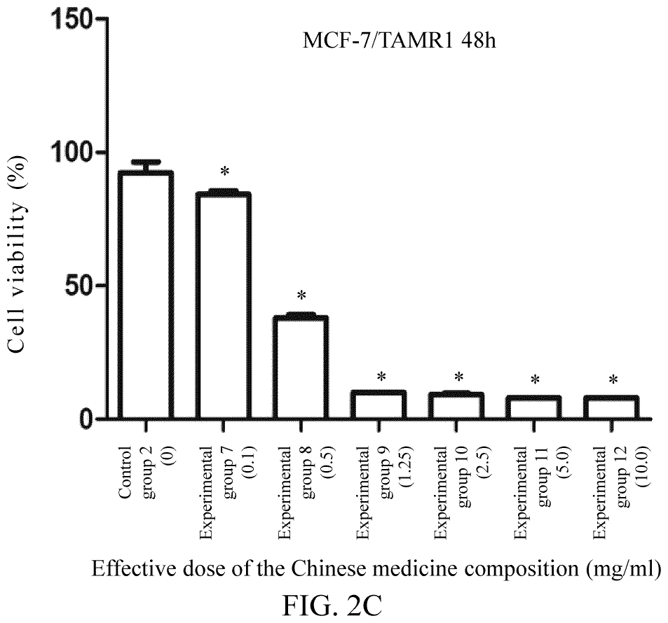

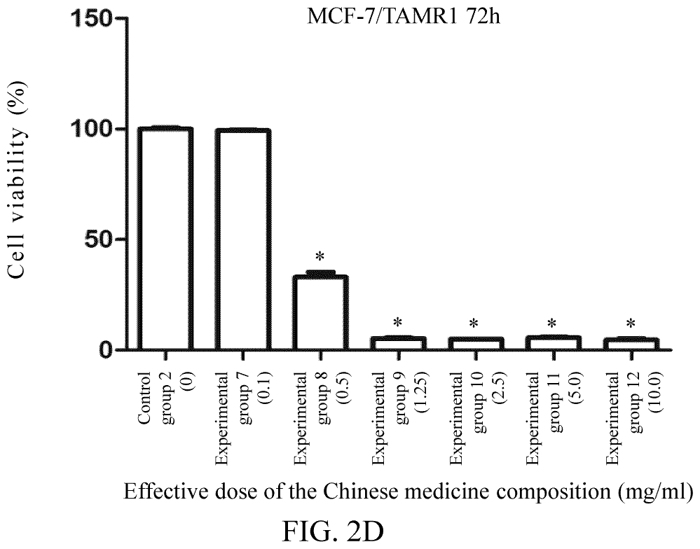

The observation results are shown in FIG. 2A, FIG. 2B, FIG. 2C, and FIG. 2D. FIG. 2A is a chart displaying a plurality of cell images, showing the tamoxifen-resistant breast cancer cells (MCF-7/TAMR 1) in the experimental groups 7-12, corresponding to 24-hour, 48-hour, and 72-hour cultivation times. FIG. 2B, FIG. 2C, and FIG. 2D respectively present bar charts, showing cell viability data of the tamoxifen-resistant breast cancer cells (MCF-7/TAMR 1) in the experimental groups 7-12, corresponding to 24-hour, 48-hour, and 72-hour cultivation times. In FIG. 2B to FIG. 2D, the Standard Error (SE) for the experimental examples 7-12 is shown, which were obtained from three independent tests (n=3) conducted for each of the experimental groups 7-12. The symbol “*” indicates a statistically significant comparison between the experimental groups 7-12 and the control group 2, with p<0.05.

As shown in FIG. 2A and FIG. 2B, based on the results from the experimental groups 7-12 under 24-hour cultivation time, the cell viability of the tamoxifen-resistant breast cancer cells (MCF-7/TAMR1) in the experimental groups 7 and 8 showed a gradual decrease compared to the control group 2. This indicates that administering the effective doses of 0.1 mg/ml and 0.5 mg/ml of the Chinese medicine composition in the experimental groups 7 and 8, respectively, could appropriately inhibit or reduce the growth of the tamoxifen-resistant breast cancer cells (MCF-7/TAMR1). The cell viability of the tamoxifen-resistant breast cancer cells (MCF-7/TAMR1) in the experimental groups 9-12 was significantly reduced to below 10% compared to the control group 2. As shown in FIG. 2A, the cell images of the experimental groups 9-12 showed a significant reduction in cell density compared to the experimental groups 7 and 8. This indicates that administering the effective doses ranging from 1.25 mg/ml to 10.0 mg/ml of the Chinese medicine composition under 24-hour cultivation time in the experimental groups 9-12 effectively reduced or inhibited the growth of the tamoxifen-resistant breast cancer cells (MCF-7/TAMR1).

As shown in FIG. 2A and FIG. 2C, based on the results from the experimental groups 7-12 under 48-hour cultivation time, the cell viability of the tamoxifen-resistant breast cancer cells (MCF-7/TAMR1) in the experimental groups 7 and 8showed a decreasing trend compared to the control group 2. As shown in FIG. 2B and FIG. 2C, the cell viability of the tamoxifen-resistant breast cancer cells (MCF-7/TAMR1) in the experimental group 8 was further reduced under 48-hour cultivation time compared to 24-hour cultivation time. The cell viability of the tamoxifen-resistant breast cancer cells (MCF-7/TAMR1) in the experimental group 8 was reduced to below 50% under 48-hour cultivation time. The cell viability of the tamoxifen-resistant breast cancer cells (MCF-7/TAMR 1) in the experimental groups 9-12 was also reduced to below 10% compared to the control group 2. The cell viability of the tamoxifen-resistant breast cancer cells (MCF-7/TAMR 1) in the experimental group 9-12 was slightly reduced under 48-hour cultivation time compared to 24-hour cultivation time.

As shown in FIG. 2A and FIG. 2D, based on the results from the experimental groups 7-12 under 72-hour cultivation time, the cell viability of the tamoxifen-resistant breast cancer cells (MCF-7/TAMR1) in the experimental group 7 did not significantly decrease compared to the control group 2. The cell viability of the tamoxifen-resistant breast cancer cells (MCF-7/TAMR1) in the experimental group 8 was reduced to below 50%. The cell viability of the tamoxifen-resistant breast cancer cells (MCF-7/TAMR 1) in the experimental groups 9-12 was also reduced to below 10% compared to the control group 2. The cell viability of the tamoxifen-resistant breast cancer cells (MCF-7/TAMR 1) in the experimental group 9-12 was slightly reduced under 72-hour cultivation time compared to 48-hour cultivation time.

Thus, the effective doses of the Chinese medicine composition ranging from 0.1 to 10.0 mg/ml could inhibite or reduce the activity of the tamoxifen-resistant breast cancer cells (MCF-7/TAMR 1). The effective doses ranging from 0.5 to 10.0 mg/ml could effectively reduce the cell viability of the the tamoxifen-resistant breast cancer cells (MCF-7/TAMR1). The effective doses of the Chinese medicine composition ranging from 1.25 to 10.0 mg/ml more significantly inhibit the activity of the tamoxifen-resistant breast cancer cells (MCF-7/TAMR1).

The following investigates the effects of the experimental groups 7-12 on inhibiting the migration of the tamoxifen-resistant breast cancer cells (MCF-7/TAMR1):

A migration assay includes the following steps: preparing a plate having 24 wells and a plurality of hanging cell culture inserts (EMD Millipore Corporation, USA) disposed in the respective wells, wherein each cell culture insert includes an upper chamber and a lower chamber; for each upper chamber, placing the tamoxifen-resistant breast cancer cells (MCF-7/TAMR1) in each upper chamber of the cell culture inserts, adding different effective doses of the Chinese medicine composition prepared for the experimental groups 7-12 into each upper chamber, respectively, and inserting serum-free medium into each upper chamber to limit cell growth; for each lower chamber, inserting standard culture medium into each lower chamber to attract the tamoxifen-resistant breast cancer cells (MCF-7/TAMR1); combining the upper chamber and the lower chamber of each cell culture insert; placing the plate and the cell culture inserts in a 37° C. incubator for 24 hours; removing the tamoxifen-resistant breast cancer cells (MCF-7/TAMR1) that did not migrate from the upper chambers of each cell culture insert; fixing the tamoxifen-resistant breast cancer cells (MCF-7/TAMR1) that migrated into the lower chambers of each cell culture insert with 4% paraformaldehyde; staining the tamoxifen-resistant breast cancer cells (MCF-7/TAMR1) that migrated into the lower chambers of each cell culture insert with 0.05% Giemsa stain; observing and analyzing the number of migrated tamoxifen-resistant breast cancer cells (MCF-7/TAMR1).

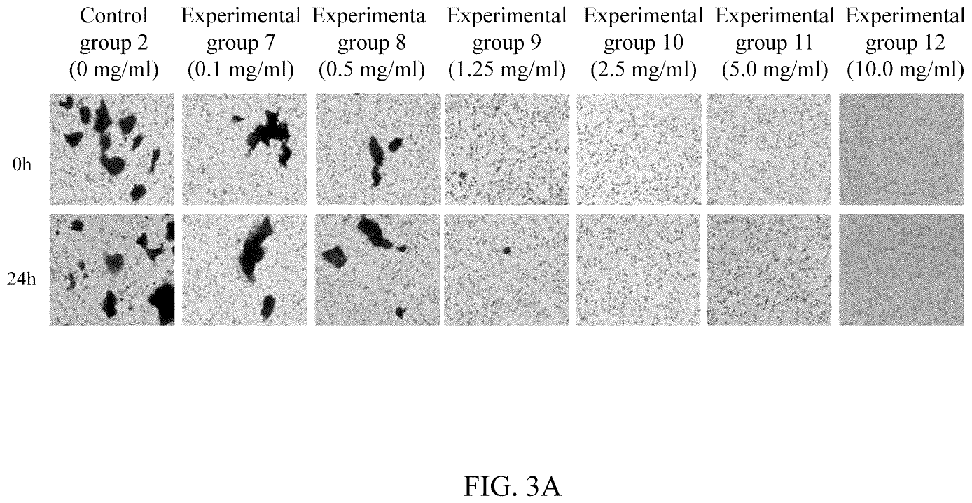

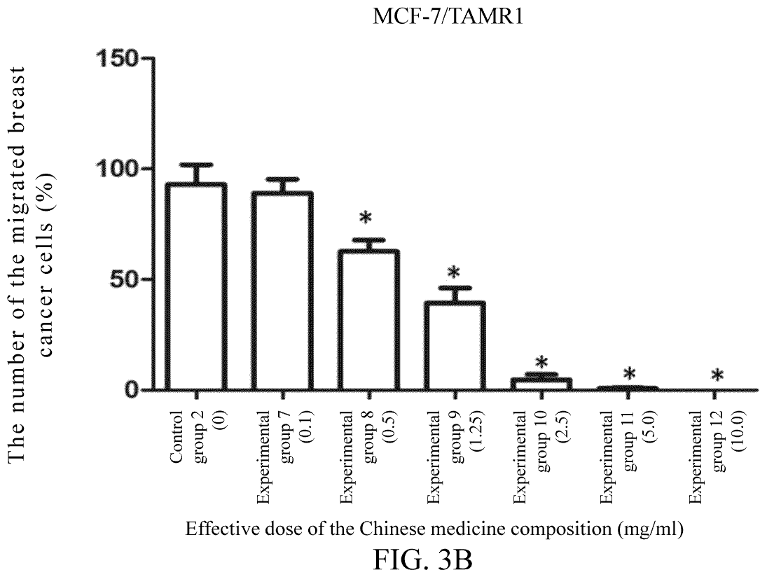

The observation results are shown in FIG. 3A and FIG. 3B. FIG. 3A is a chart displaying a plurality of cell images, showing the tamoxifen-resistant breast cancer cells (MCF-7/TAMR 1) in the experimental groups 7-12 under 24-hour cultivation time in the migration assay. FIG. 3B is a bar chart, showing the cell migration data of the tamoxifen-resistant breast cancer cells (MCF-7/TAMR1) in the experimental groups 7-12. In FIG. 3B, the Standard Error (SE) for the experimental examples 7-12 is shown, which were obtained from three independent tests (n=3) conducted for each of the experimental groups 7-12. The symbol “*” indicates a statistically significant comparison between the experimental groups 7-12 and the control group 2, with p<0.05.

As shown in FIG. 3A and FIG. 3B, based on the results from the experimental groups 7-12 under 24-hour cultivation time, the number of migrated tamoxifen-resistant breast cancer cells (MCF-7/TAMR1) in the experimental group 7 showed no significant difference compared to the control group 2, indicating that administering the effective dose of 0.1 mg/ml of the Chinese medicine composition in the experimental group 7 was unable to effectively inhibit the cell migration of the tamoxifen-resistant breast cancer cells (MCF-7/TAMR1). The numbers of the migrated tamoxifen-resistant breast cancer cells (MCF-7/TAMR1) in the experimental groups 8 and 9 significantly decreased compared to the control group 2. The numbers of the migrated tamoxifen-resistant breast cancer cells (MCF-7/TAMR1) in the experimental groups 10-12 was significantly reduced to below 10% compared to the control group 2. This indicates that administering the effective doses ranging from 2.5 mg/ml to 10.0 mg/ml of the Chinese medicine composition under 24-hour cultivation time in the experimental groups 10-12 effectively inhibits the cell migration of the tamoxifen-resistant breast cancer cells (MCF-7/TAMR1).

The following investigates the effects of the experimental groups 7-12 on inhibiting the invasion of the tamoxifen-resistant breast cancer cells (MCF-7/TAMR1):

An invasion assay follows steps similar to the steps of the migration assay. The invasion assay includes the following steps: preparing a plurality of hanging cell culture inserts, wherein each cell culture insert includes an upper chamber and a lower chamber. For each upper chamber, coating 0.4 mg/mL of the bio-matrix (Matrigel) on a membrane of the upper chamber of each cell culture insert; inoculating the tamoxifen-resistant breast cancer cells (MCF-7/TAMR1) onto each bio-matrix; and adding different effective doses of the Chinese medicine composition prepared for the experimental groups 7-12 onto the membrane of each upper chamber. Thus, the barrier in the organ tissue is simulated, requiring the tamoxifen-resistant breast cancer cells (MCF-7/TAMR1) not only to migrate but also to possess the ability to invade the bio-matrix. For each lower chamber, inserting standard culture medium into each lower chamber to attract the tamoxifen-resistant breast cancer cells (MCF-7/TAMR1); combining the upper chamber and the lower chamber of each cell culture insert; placing the cell culture inserts in a 37° C. incubator for 24 hours; removing the tamoxifen-resistant breast cancer cells (MCF-7/TAMR1) that did not migrate from the upper chambers of each cell culture insert; fixing the tamoxifen-resistant breast cancer cells (MCF-7/TAMR1) that migrated into the lower chambers of each cell culture insert with 4% paraformaldehyde; staining the tamoxifen-resistant breast cancer cells (MCF-7/TAMR1) that migrated into the lower chambers of each cell culture insert with 0.05% Giemsa stain; observing and analyzing the number of the tamoxifen-resistant breast cancer cells (MCF-7/TAMR1) that underwent cell invasion and subsequent migration.

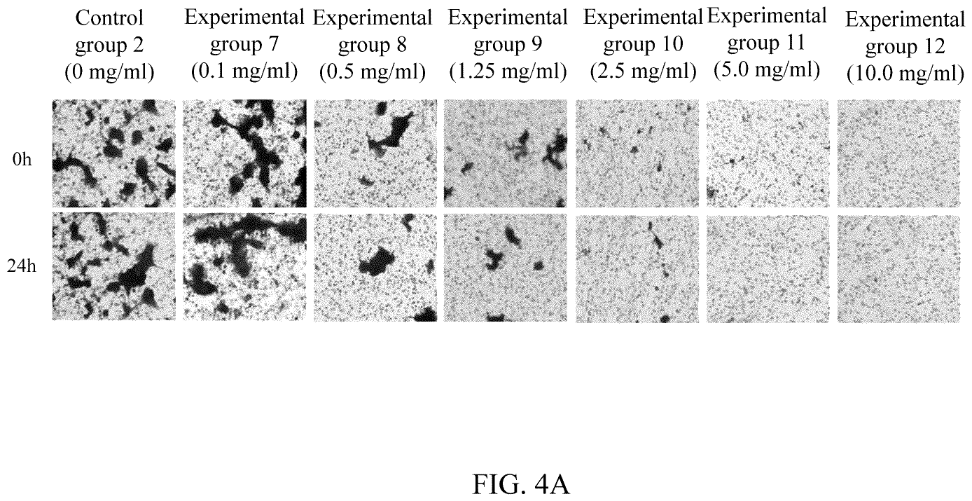

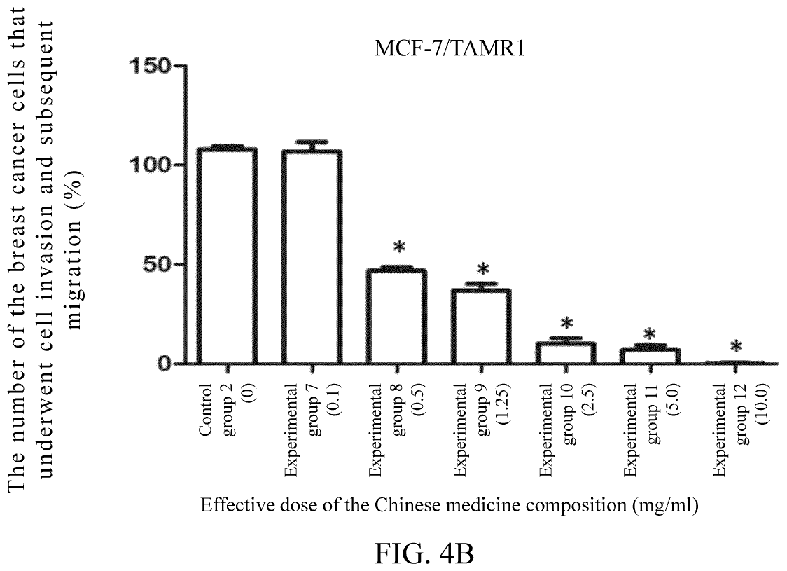

As shown in FIG. 4A and FIG. 4B, the number of the tamoxifen-resistant breast cancer cells (MCF-7/TAMR1) that underwent cell invasion and subsequent migration in the experimental group 7 showed no significant difference compared to the control group 2, indicating that administering the effective dose of 0.1 mg/ml of the Chinese medicine composition in the experimental group 7 was unable to effectively inhibit the cell invasion of the tamoxifen-resistant breast cancer cells (MCF-7/TAMR1). The numbers of the tamoxifen-resistant breast cancer cells (MCF-7/TAMR1) that underwent cell invasion and subsequent migration in the experimental groups 8 and 9 was significantly reduced to below 50% compared to the control group 2. The numbers of the tamoxifen-resistant breast cancer cells (MCF-7/TAMR1) that underwent cell invasion and subsequent migration in the experimental groups 10-12 was significantly reduced to below 10% compared to the control group 2. This indicates that administering the effective doses ranging from 2.5 mg/ml to 10.0 mg/ml of the Chinese medicine composition under 24-hour cultivation time in the experimental groups 10-12 effectively inhibits the cell invasion of the tamoxifen-resistant breast cancer cells (MCF-7/TAMR1).

The following investigates the autophagy effect of the tamoxifen-resistant breast cancer cells (MCF-7/TAMR1) corresponding to the administration of the experimental groups 7-1:

Western blot (autophagic cell activity assay):

The tamoxifen-resistant breast cancer cells (MCF-7/TAMR1) in the experimental groups 7-12 were cultured for 24 hours, respectively, to detect the expression of ATG5, ATG7, and LC3-II proteins in autophagy using antibodies targeting ATG5, ATG7, and LC3-II. First, incubating the polyvinylidene difluoride (PVDF) membrane with antibodies targeting ATG5, ATG7, and LC3-II proteins for 3 hours, followed by washing with phosphate-buffered saline (PBS). Then, adding horse-radish peroxidase (HRP) antibody and incubating for 1 hour. Finally, detecting the antigen-antibody complex using Immobilion Western HRP chemiluminescent substrate and measuring protein expression with a densitometer (GE ImageQuant TL 8.1; GE Healthcare Life Sciences).

Additionally, autophagy is a physiological process within cells, primarily responsible for degrading old proteins, damaged organelles, and foreign microorganisms. Numerous proteins are involved in regulating the autophagy process. The microtubule-associated protein 1A/1B-light chain 3(LC3 ) in the cytoplasm is often used as a fluorescent marker for autophagy activation. When autophagy is activated, LC3 binds with the lipid phosphatidylethanolamine to form LC3-II protein, which then integrates into the membrane of the phagosome. Therefore, the number of LC3-II protein generated inside the vesicle serves as an indicator of autophagy activation.

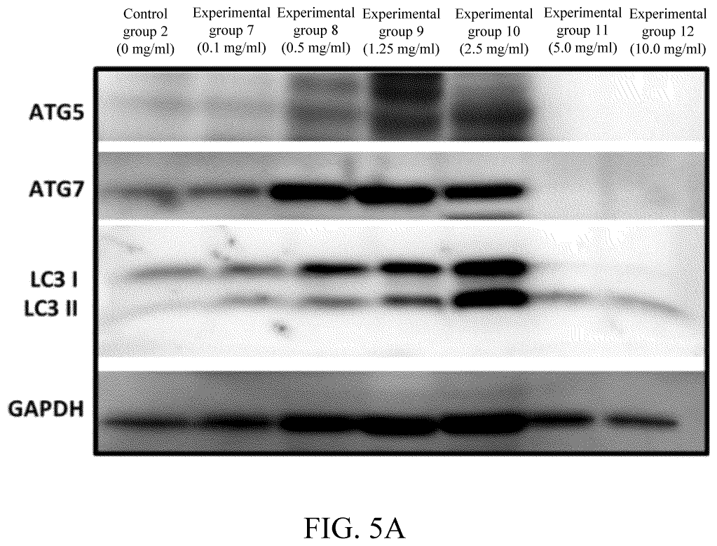

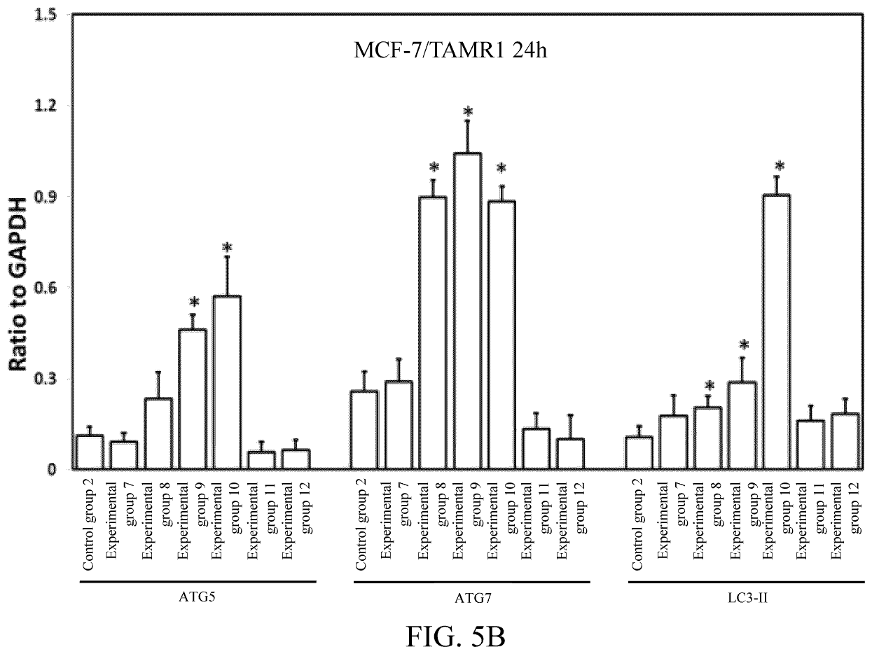

The observation results are shown in FIG. 5A and FIG. 5B. The expression of LC3-II protein in the experimental groups 8-10 was significantly increased compared to the control group 2. This indicates that administering the effective doses ranging from 0.5 mg/ml to 2.5 mg/ml of the Chinese medicine composition under in the experimental groups 8-10 significantly induce LC3-II protein expression in the tamoxifen-resistant breast cancer cells (MCF-7/TAMR1). Among the experimental groups 8-10, the experimental group 10 showed the most significant increase in LC3-II protein expression. Therefore, the Chinese medicine composition, at the specific effective doses, could effectively trigger autophagy in the tamoxifen-resistant breast cancer cells (MCF-7/TAMR1), achieving significant inhibition and reduction of the activity of the the tamoxifen-resistant breast cancer cells (MCF-7/TAMR1). In the experimental groups 11 and 12, the LC3-II protein expression in the tamoxifen-resistant breast cancer cells (MCF-7/TAMR1) showed evidence of degradation, indicating that the effective doses of 5 mg/ml and 10 mg/ml of the Chinese medicine composition in the experimental groups 11 and 12 significantly activated LC3-II protein and induced autophagy in the tamoxifen-resistant breast cancer cells (MCF-7/TAMR1). As shown in FIG. 2B, the cell activity of the tamoxifen-resistant breast cancer cells (MCF-7/TAMR1) in the experimental groups 11 and 12 significantly decreased. After the tamoxifen-resistant breast cancer cells (MCF-7/TAMR1) underwent autophagy, the effective doses of the Chinese medicine composition increased the expression of ATG5, ATG7 proteins, and the degradation of LC3-II protein.

The following investigates the cell viability of the tamoxifen-sensitive breast cancer cells (MCF-7/S0.5) after administering the Chinese medicine composition:

1. The groups in the current test include control group 3 and experimental groups 13 to 18.

Control group 3: The control group 3 includes the tamoxifen-sensitive breast cancer cells (MCF-7/S0.5) that were not administered the Chinese medicine composition. An effective dose of the Chinese medicine composition is 0 mg/ml. The tamoxifen-sensitive breast cancer cells (MCF-7/S0.5) were cultured for 24, 48, and 72 hours, respectively.

Experimental group 13: The experimental group 13 includes the tamoxifen-sensitive breast cancer cells (MCF-7/S0.5) that were administered the Chinese medicine composition at an effective dose of 0.1 mg/ml. The tamoxifen-sensitive breast cancer cells (MCF-7/S0.5) were cultured for 24, 48, and 72 hours, respectively, following the administration.

Experimental group 14: The experimental group 14 includes the tamoxifen-sensitive breast cancer cells (MCF-7/S0.5) that were administered the Chinese medicine composition at an effective dose of 0.5 mg/ml. The tamoxifen-sensitive breast cancer cells (MCF-7/S 0.5) were cultured for 24, 48, and 72 hours, respectively, following the administration.

Experimental group 15: The experimental group 15 includes the tamoxifen-sensitive breast cancer cells (MCF-7/S0.5) that were administered the Chinese medicine composition at an effective dose of 1.25 mg/ml. The tamoxifen-sensitive breast cancer cells (MCF-7/S0.5) were cultured for 24, 48, and 72 hours, respectively, following the administration.

Experimental group 16: The experimental group 16 includes the tamoxifen-sensitive breast cancer cells (MCF-7/S0.5) that were administered the Chinese medicine composition at an effective dose of 2.5 mg/ml. The tamoxifen-sensitive breast cancer cells (MCF-7/S 0.5) were cultured for 24, 48, and 72 hours, respectively, following the administration.

Experimental group 17: The experimental group 17 includes the tamoxifen-sensitive breast cancer cells (MCF-7/S0.5) that were administered the Chinese medicine composition at an effective dose of 5.0 mg/ml. The tamoxifen-sensitive breast cancer cells (MCF-7/S 0.5) were cultured for 24, 48, and 72 hours, respectively, following the administration.

Experimental group 18: The experimental group 18 includes the tamoxifen-sensitive breast cancer cells (MCF-7/S0.5) that were administered the Chinese medicine composition at an effective dose of 10.0 mg/ml. The tamoxifen-sensitive breast cancer cells (MCF-7/S0.5) were cultured for 24, 48, and 72 hours, respectively, following the administration.

2. Cell viability analysis of the tamoxifen-sensitive breast cancer cells (MCF-7/S0.5) in each of the experimental groups 1-6:

The cell viability test steps for the tamoxifen-sensitive breast cancer cells (MCF-7/S0.5) in the control group 3 and the experimental groups 13-18 are identical to the cell viability test steps in the control group 1 and the experimental groups 1-6, and are not reiterated here.

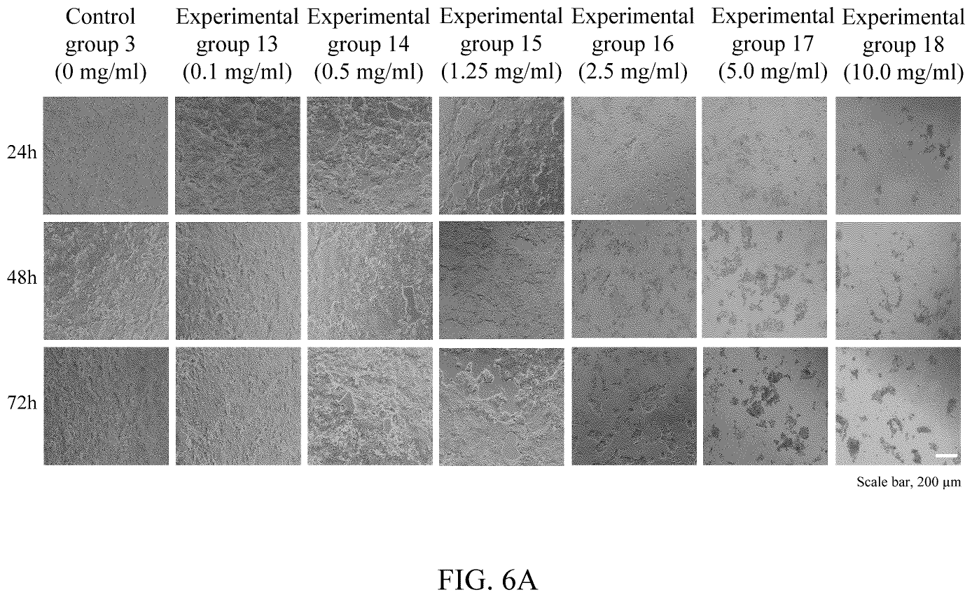

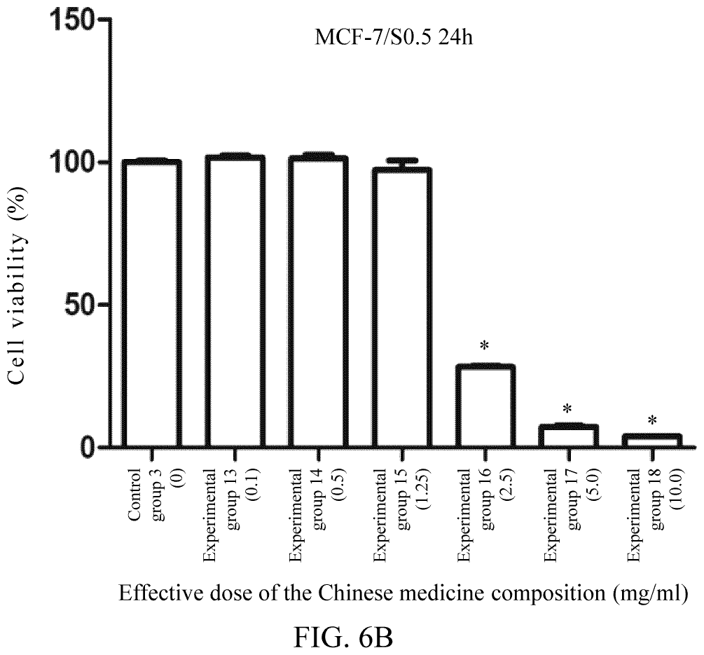

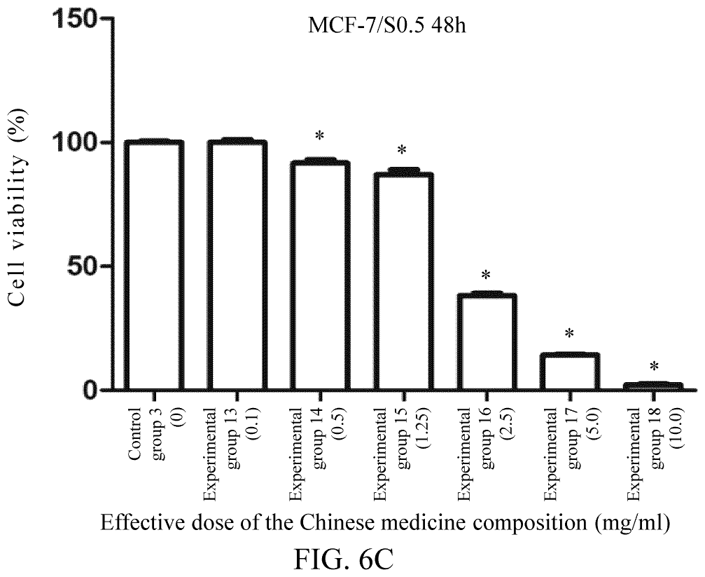

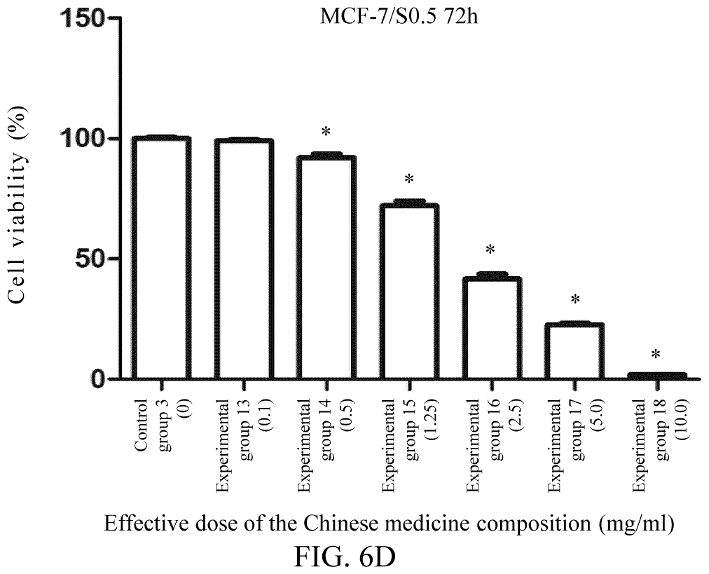

The observation results are shown in FIG. 6A, FIG. 6B, FIG. 6C, and FIG. 6D. FIG. 6A is a chart displaying a plurality of cell images, showing the tamoxifen-sensitive breast cancer cells (MCF-7/S 0.5) in the experimental groups 13-18 , corresponding to 24-hour, 48-hour, and 72-hour cultivation times. FIG. 6B, FIG. 6C, and FIG. 6D respectively present bar charts, showing cell viability data of the tamoxifen-sensitive breast cancer cells (MCF-7/S 0.5) in the experimental groups 13-18 , corresponding to 24-hour, 48-hour, and 72-hour cultivation times. In FIG. 6B to FIG. 6D, the Standard Error (SE) for the experimental examples 13-18 is shown, which were obtained from three independent tests (n=3) conducted for each of the experimental groups 13-18. The symbol “*” indicates a statistically significant comparison between the experimental groups 13-18 and the control group 3, with p<0.05.

As shown in FIG. 6A and FIG. 6B, based on the results from the experimental groups 13-18 under 24-hour cultivation time, the cell viability of the tamoxifen-sensitive breast cancer cells (MCF-7/S0.5) in the experimental groups 13-15 did not show a significant decrease compared to the control group 3. The cell viability of the tamoxifen-sensitive breast cancer cells (MCF-7/S0.5) in the experimental groups 16-18 was significantly reduced to below 50%. The cell viability of the tamoxifen-sensitive breast cancer cells (MCF-7/S0.5) in the experimental groups 17 and 18 was significantly reduced to below 10%, wherein the cell viability of the tamoxifen-sensitive breast cancer cells (MCF-7/S0.5) in the experimental group 18 was further reduced compared to the experimental group 17. As shown in FIG. 6A, the cell images of the experimental groups 16-18 showed a significant reduction in cell density compared to the experimental groups 13-15. This indicates that administering the effective doses ranging from 2.5 mg/ml to 10.0 mg/ml of the Chinese medicine composition under 24-hour cultivation time in the experimental groups 16-18 effectively reduced or inhibited the growth of the tamoxifen-sensitive breast cancer cells (MCF-7/S0.5). Among the experimental groups 16-18, the effective dose of the Chinese medicine composition administered in the experimental group 18, 10.0 mg/ml, demonstrated greater effectiveness in inhibiting the growth of the tamoxifen-sensitive breast cancer cells (MCF-7/S0.5).

As shown in FIG. 6A and FIG. 6C, based on the results from the experimental groups 13-18 under 48-hour cultivation time, the cell viability of the tamoxifen-sensitive breast cancer cells (MCF-7/S0.5) in the experimental group 13 also did not show a significant decrease. The cell viability of the tamoxifen-sensitive breast cancer cells (MCF-7/S0.5) in the experimental groups 14 and 15 showed a gradual decrease compared to the control group 3. This indicates that administering the effective doses of 0.5 mg/ml and 1.25 mg/ml of the Chinese medicine composition in the experimental groups 14 and 15, respectively, under 48-hour cultivation time, could appropriately inhibit or reduce the growth of the tamoxifen-sensitive breast cancer cells (MCF-7/S0.5). The cell viability of the tamoxifen-sensitive breast cancer cells (MCF-7/S0.5) in the experimental groups 16-18 was significantly reduced to below 50%. The cell viability of the tamoxifen-sensitive breast cancer cells (MCF-7/S 0.5) in the experimental group 18 was further reduced under 48-hour cultivation time compared to 24-hour cultivation time.

As shown in FIG. 6A and FIG. 6D, based on the results from the experimental groups 13-18 under 72-hour cultivation time, the cell viability of the tamoxifen-sensitive breast cancer cells (MCF-7/S0.5) in the experimental group 13 also did not show a significant decrease. The cell viability of the tamoxifen-sensitive breast cancer cells (MCF-7/S0.5) in the experimental groups 14 and 15 showed a further decrease compared to the control group 3. As shown in FIG. 6C and FIG. 6D, the cell viability of the tamoxifen-sensitive breast cancer cells (MCF-7/S0.5) in the experimental group 15 was further reduced under 72-hour cultivation time compared to 48-hour cultivation time. The cell viability of the tamoxifen-sensitive breast cancer cells (MCF-7/S0.5) in the experimental groups 16-18 was significantly reduced to below 50%.

Thus, the effective doses of the Chinese medicine composition ranging from 0.5 to 10.0 mg/ml could effectively inhibite or reduce the activity of the tamoxifen-sensitive breast cancer cells (MCF-7/S 0.5). The effective doses ranging from 2.5 to 10.0 mg/ml could significantly reduce the cell viability of the the tamoxifen-sensitive breast cancer cells (MCF-7/S 0.5). The effective doses of the Chinese medicine composition ranging from 5.0 to 10.0 mg/ml more significantly inhibit the activity of tamoxifen-sensitive breast cancer cells (MCF-7/S0.5).

The following investigates the effects of the experimental groups 13-18 on inhibiting the migration of the tamoxifen-sensitive breast cancer cells (MCF-7/S0.5):

The steps of a migration assay for the tamoxifen-sensitive breast cancer cells (MCF-7/S0.5) in the control group 3 and the experimental groups 13-18 are identical to the steps of the migration assay in the control group 2 and the experimental groups 7-13, and are not reiterated here.

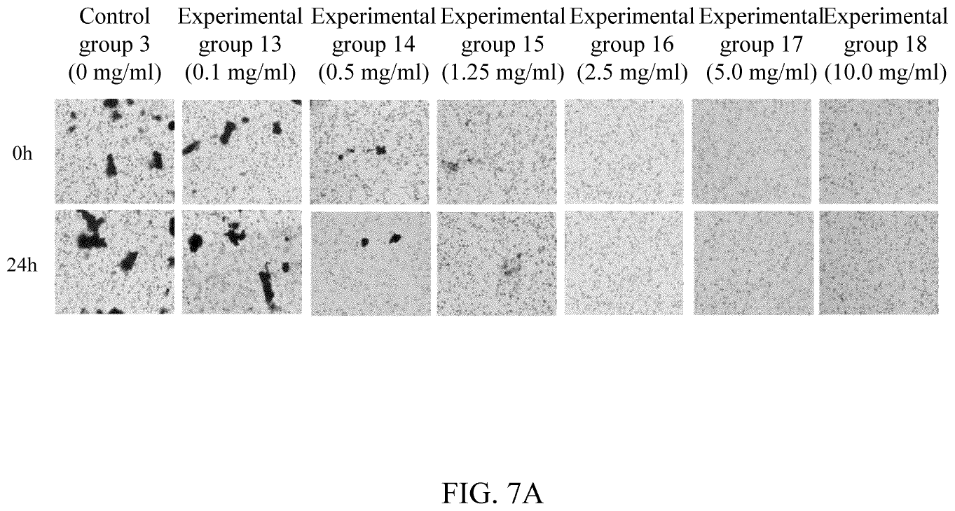

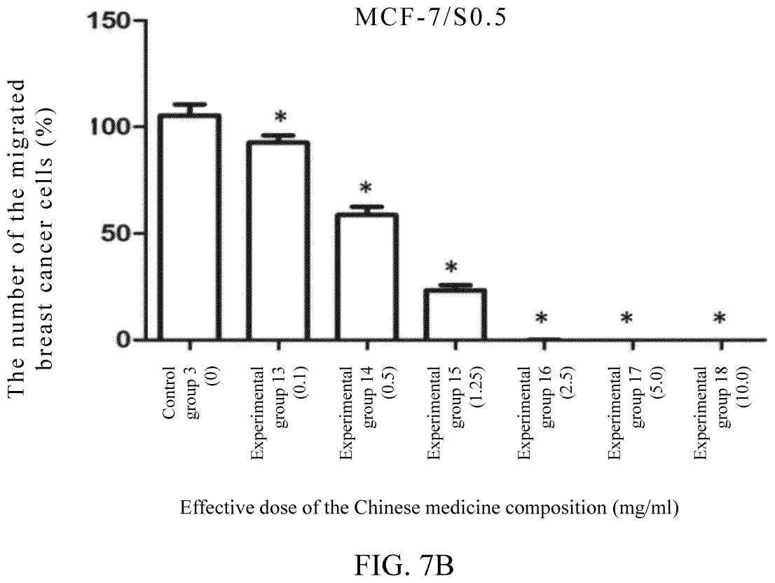

The observation results are shown in FIG. 7A and FIG. 7B. FIG. 7A is a chart displaying a plurality of cell images, showing the tamoxifen-sensitive breast cancer cells (MCF-7/S 0.5) in the experimental groups 13-18 under 24-hour cultivation time in the migration assay. FIG. 7B is a bar chart, showing the cell migration data of the tamoxifen-sensitive breast cancer cells (MCF-7/S0.5) in the experimental groups 13-18 . In FIG. 7B, the Standard Error (SE) for the experimental examples 13-18 is shown, which were obtained from three independent tests (n=3) conducted for each of the experimental groups 13-18 . The symbol “*” indicates a statistically significant comparison between the experimental groups 13-18 and the control group 3, with p<0.05.

As shown in FIG. 7A and FIG. 7B, based on the results from the experimental groups 13-18 under 24-hour cultivation time, the number of migrated tamoxifen-sensitive breast cancer cells (MCF-7/S0.5) in the experimental group 13 showed a slight decrease compared to the control group 3. The numbers of the migrated tamoxifen-sensitive breast cancer cells (MCF-7/S0.5) in the experimental groups 14 and 15 was significantly reduced compared to the control group 3. The number of migrated tamoxifen-sensitive breast cancer cells (MCF-7/S0.5) in the experimental groups 16-18 was significantly reduced to below 10% compared to the control group 3. This indicates that administering the effective doses ranging from 2.5 mg/ml to 10.0 mg/ml of the Chinese medicine composition under 24-hour cultivation time in the experimental groups 16-18 effectively inhibits the cell migration of the tamoxifen-sensitive breast cancer cells (MCF-7/S0.5).

The following investigates the effects of the experimental groups 13-18 on inhibiting the invasion of the tamoxifen-sensitive breast cancer cells (MCF-7/S0.5):

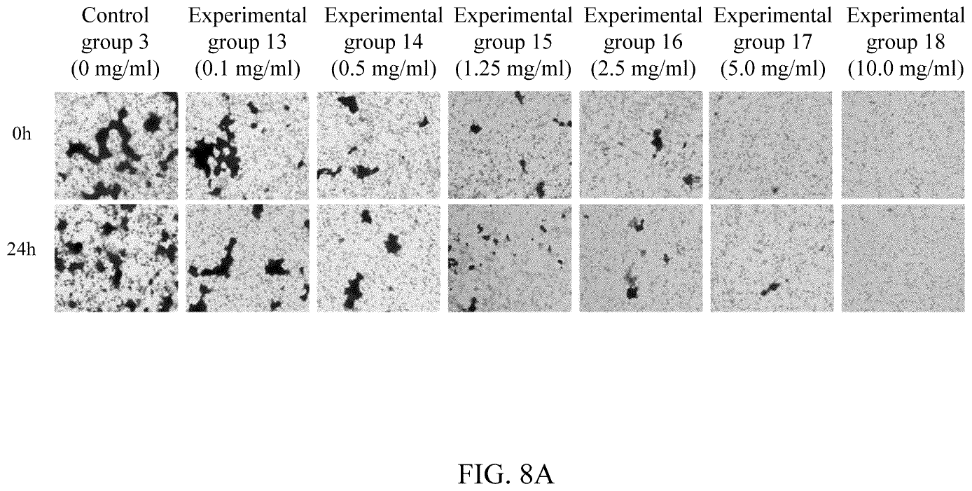

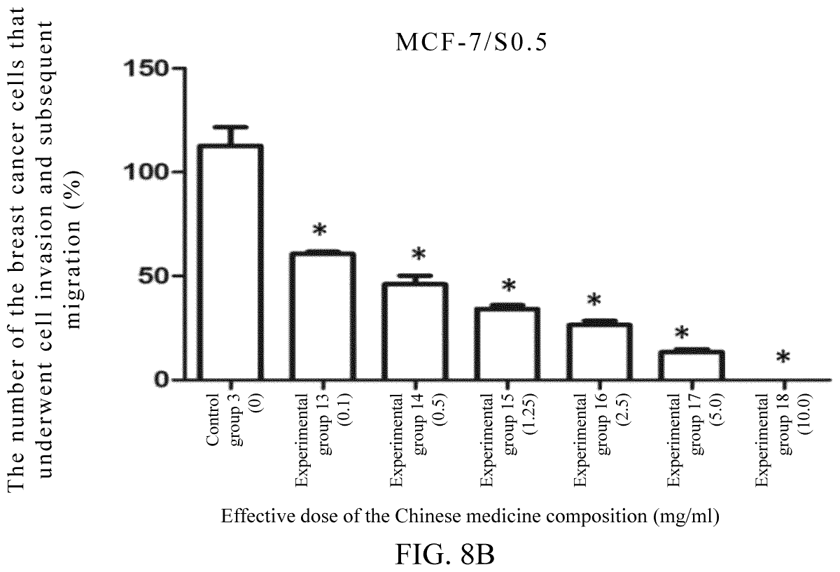

As shown in FIG. 8A and FIG. 8B, the number of the tamoxifen-sensitive breast cancer cells (MCF-7/S0.5) that underwent cell invasion and subsequent migration in the experimental groups 13-18 was significantly reduced compared to the control group 3. The numbers of the tamoxifen-sensitive breast cancer cells (MCF-7/S0.5) that underwent cell invasion and subsequent migration in the experimental groups 14-18 was significantly reduced to below 50%. The numbers of the tamoxifen-sensitive breast cancer cells (MCF-7/S 0.5) that underwent cell invasion and subsequent migration in the experimental group 18 was significantly reduced to below 10%. This indicates that administering the effective doses ranging from 0.1 mg/ml to 10.0 mg/ml of the Chinese medicine composition under 24-hour cultivation time in the experimental groups 13-18 effectively inhibits the cell invasion of the tamoxifen-sensitive breast cancer cells (MCF-7/S0.5).

The following investigates the autophagy effect of the tamoxifen-sensitive breast cancer cells (MCF-7/S0.5) corresponding to the administration of the experimental groups 13-18:

The steps of Western blot and the detection of the expression of ATG5, ATG7, and LC3-II proteins in the tamoxifen-sensitive breast cancer cells (MCF-7/S0.5) in the control group 3 and the experimental groups 13-18 are identical to those in the control group 2 and the experimental groups 7-13, and are not reiterated here.

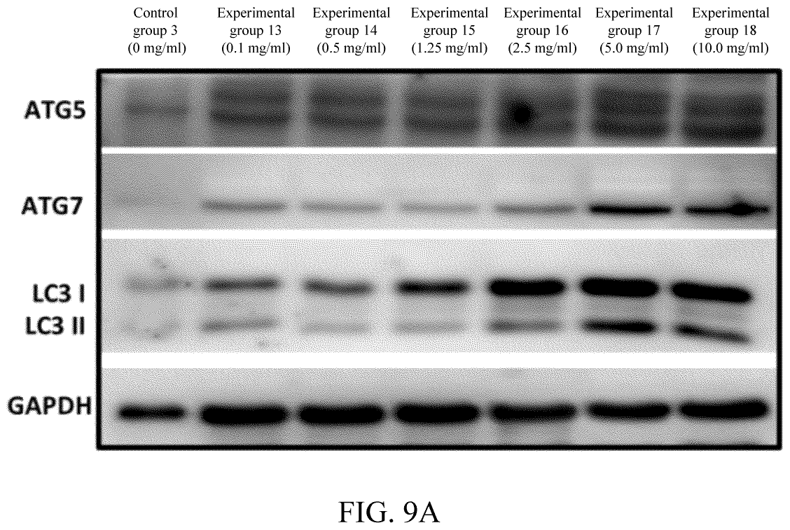

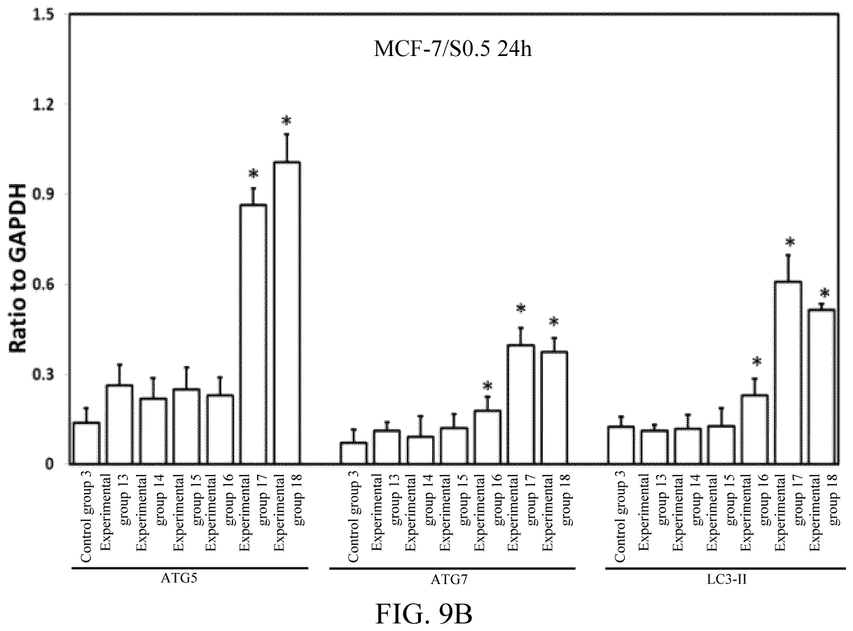

The observation results are shown in FIG. 9A and FIG. 9B. The expression of LC3-II protein in the experimental groups 16-18 was significantly increased compared to the control group 3. This indicates that administering the effective doses ranging from 2.5 mg/ml to 10.0 mg/ml of the Chinese medicine composition under in the experimental groups 16-18 significantly induce LC3-II protein expression in the tamoxifen-sensitive breast cancer cells (MCF-7/S0.5). Experimental groups 16-18 significantly induced LC3-II protein expression in the tamoxifen-sensitive breast cancer cells (MCF-7/S0.5), thus significantly decreasing cell viability to below 50% in the experimental groups 16-18, as shown in FIG. 6B. As shown in FIG. 9B, the LC3-II protein expression detected in experimental group 17 was the most significant compared to experimental groups 16 and 18. In summary, the Chinese medicine composition, at the specific effective doses, effectively induces autophagy in the tamoxifen-sensitive breast cancer cells (MCF-7/S0.5), achieving inhibition and reduction of the activity of the the tamoxifen-sensitive breast cancer cells (MCF-7/S0.5).

In summary, the weight ratios of Euphorbia kansui, Paeonia lactiflora, Glycyrrhiza uralensis, Taraxacum, Gardeniae fructus, and Hordeum vulgare in the Chinese medicine composition have been validated through the cell tests to effectively inhibit or reduce the activity of the breast cancer cells. The effective dose of the Chinese medicine composition, ranging from 0.1 to 10.0 mg/ml, could effectively inhibit the activity of the conventional breast cancer cells (MCF-7), the tamoxifen-resistant breast cancer cells (MCF-7/TAMR1), and the tamoxifen-sensitive breast cancer cells (MCF-7/S 0.5) under at least 24 hours of cultivation time. Correspondingly, the effective dose of the Chinese medicine composition, ranging from 0.1 to 10.0 mg/ml, reduces the number of the conventional breast cancer cells (MCF-7), the tamoxifen-resistant breast cancer cells (MCF-7/TAMR1), and the tamoxifen-sensitive breast cancer cells (MCF-7/S0.5). When the breast cancer cells are the conventional breast cancer cells (MCF-7), the effective dose of the Chinese medicine composition, ranging from 5.0 to 10.0 mg/ml, effectively inhibits the activity of the conventional breast cancer cells (MCF-7). When the breast cancer cells are the tamoxifen-resistant breast cancer cells (MCF-7/TAMR1), the effective dose of the Chinese medicine composition, ranging from 1.25 to 10.0 mg/ml, effectively inhibits the activity of the tamoxifen-resistant breast cancer cells (MCF-7/TAMR1). When the breast cancer cells are the tamoxifen-sensitive breast cancer cells (MCF-7/S0.5), the effective dose of the Chinese medicine composition, ranging from 2.5 to 10.0 mg/ml, effectively inhibits the activity of the tamoxifen-sensitive breast cancer cells (MCF-7/S0.5). Therefore, the composition ratio of the Chinese medicine composition, when administered at the specific effective dose, achieves the goal of treating breast cancer symptoms or inhibiting the activity of breast cancer cells.

It must be pointed out that the embodiment described above is only a preferred embodiment of the present invention. All equivalent formulas and methods which employ the concepts disclosed in this specification and the appended claims should fall within the scope of the present invention.

What is claimed is:

1. A Chinese medicine composition for treating breast cancer, comprising
Euphorbia kansui, Paeonia lactiflora, Glycyrrhiza uralensis, Taraxacum, Gardeniae fructus, and Hordeum vulgare;
wherein a weight ratio of Euphorbia kansui, Paeonia lactiflora, Glycyrrhiza uralensis, Taraxacum, Gardeniae fructus, and Hordeum vulgare in the Chinese medicine composition is 1-2:4-6:2-3:3-5:2-4:2-3.

2. The Chinese medicine composition as claimed in claim 1, wherein the Chinese medicine composition is for inhibiting or reducing an activity of a plurality of breast cancer cells; the breast cancer cells are selected from a group consisting of conventional breast cancer cells (MCF-7), tamoxifen-resistant breast cancer cells (MCF-7/TAMR1), and tamoxifen-sensitive breast cancer cells (MCF-7/S0.5).

3. A method for treating breast cancer using a Chinese medicine composition, comprising a step of:
administering the Chinese medicine composition at an effective dose ranging from 0.1 mg/ml to 10.0 mg/ml to a plurality of breast cancer cells for cultivation;
wherein the Chinese medicine composition comprises Euphorbia kansui, Paeonia lactiflora, Glycyrrhiza uralensis, Taraxacum, Gardeniae fructus, and Hordeum vulgare; the Chinese medicine composition is for inhibiting an activity of the breast cancer cells; the breast cancer cells are selected from a group consisting of conventional breast cancer cells (MCF-7), tamoxifen-resistant breast cancer cells (MCF-7/TAMR1), and tamoxifen-sensitive breast cancer cells (MCF-7/S0.5).

4. The method for treating breast cancer using the Chinese medicine composition as claimed in claim 3, wherein a weight ratio of Euphorbia kansui, Paeonia lactiflora, Glycyrrhiza uralensis, Taraxacum, Gardeniae fructus, and Hordeum vulgare in the Chinese medicine composition is 1-2:4-6:2-3:3-5:2-4:2-3.

5. The method for treating breast cancer using the Chinese medicine composition as claimed in claim 4, wherein the breast cancer cells are the conventional breast cancer cells (MCF-7); the effective dose of the Chinese medicine composition ranges from 5.0 to 10.0 mg/ml; the Chinese medicine composition is administered to the breast cancer cells for a cultivation time ranging from 24 to 72 hours.

6. The method for treating breast cancer using the Chinese medicine composition as claimed in claim 4, wherein the breast cancer cells are the tamoxifen-resistant breast cancer cells (MCF-7/TAMR1); the effective dose of the Chinese medicine composition ranges from 0.5 to 10.0 mg/ml; the Chinese medicine composition is administered to the breast cancer cells for a cultivation time ranging from 24 to 72 hours.

7. The method for treating breast cancer using the Chinese medicine composition as claimed in claim 6, wherein the Chinese medicine composition is administered at the effective dose ranging from 0.5 to 2.5 mg/ml to the tamoxifen-resistant breast cancer cells (MCF-7/TAMR1), inducing the expression of LC3-II protein and triggers autophagy in the tamoxifen-resistant breast cancer cells (MCF-7/TAMR1).

8. The method for treating breast cancer using the Chinese medicine composition as claimed in claim 4, wherein the breast cancer cells are the tamoxifen-sensitive breast cancer cells (MCF-7/S 0.5); the effective dose of the Chinese medicine composition ranges from 2.5 to 10.0 mg/ml; the Chinese medicine composition is administered to the breast cancer cells for a cultivation time ranging from 24 to 72 hours.

9. The method for treating breast cancer using the Chinese medicine composition as claimed in claim 8, wherein the Chinese medicine composition is administered at the effective dose ranging from 2.5 to 10.0 mg/ml to the tamoxifen-sensitive breast cancer cells (MCF-7/S0.5), inducing the expression of LC3-II protein and triggers autophagy in the tamoxifen-sensitive breast cancer cells (MCF-7/S0.5).
